(19) United States
(12) Patent Application Publication
(10) Pub. No.: US 2007/0243633 A1
(43) Pub. Date: Oct. 18, 2007

(54) Biosensor

(75) Inventors: Masashi Hakamata, Kanagawa (JP); Toshiaki Kubo, Kanagawa (JP)

Correspondence Address: SUGHRUE MION, PLLC, 2100 PENNSYLVANIA AVENUE, N.W., SUITE 800, WASHINGTON, DC, 20037, US

(73) Assignee: FUJI PHOTO FILM CO., LTD.

(21) Appl. No.: 11/237,867

(22) Filed: Sep. 29, 2005

(30) Foreign Application Priority Data
Sep. 30, 2004 (JP) 285859/2004
Sep. 30, 2004 (JP) 285862/2004

(51) Int. Cl. G01N 33/543; C12M 3/00

(52) U.S. Cl. 436/518; 435/287.2; 977/902

(57) ABSTRACT

An object of the present invention is to provide a biosensor which exhibits good reproducibility of the amount of an immobilized physiologically active substance by suppressing plane deterioration (separation of a metal film from a resin) which occurs at the formation of a thin metal film layer on a resin surface. The present invention provides a biosensor which is produced by performing plasma irradiation on a light-reflecting surface of an optical block which is obtained by molding a resin and has at least 3 surfaces including a light incident surface, the light-reflecting surface and a light-emerging surface; forming a first metal layer; and then forming a second metal layer on the side of the first metal layer which is opposite to the light-reflecting surface.

### TECHNICAL FIELD

The present invention relates to a biosensor and a method for analyzing an interaction between biomolecules using the biosensor. Particularly, the present invention relates to a biosensor which is used for a surface plasmon resonance biosensor and a method for analyzing an interaction between biomolecules using the biosensor.

### BACKGROUND ART

Recently, a large number of measurements using intermolecular interactions such as immune responses are being carried out in clinical tests, etc. However, since conventional methods require complicated operations or labeling substances, several techniques are used that are capable of detecting the change in the binding amount of a test substance with high sensitivity without using such labeling substances. Examples of such a technique may include a surface plasmon resonance (SPR) measurement technique, a quartz crystal microbalance (QCM) measurement technique, and a measurement technique of using functional surfaces ranging from gold colloid particles to ultra-fine particles. The SPR measurement technique is a method of measuring changes in the refractive index near an organic functional film attached to the metal film of a chip by measuring a peak shift in the wavelength of reflected light, or changes in amounts of reflected light in a certain wavelength, so as to detect adsorption and desorption occurring near the surface. The QCM measurement technique is a technique of detecting adsorbed or desorbed mass at the ng level, using a change in frequency of a crystal due to adsorption or desorption of a substance on gold electrodes of a quartz crystal (device). In addition, the ultra-fine particle surface (nm level) of gold is functionalized, and physiologically active substances are immobilized thereon. Thus, a reaction to recognize specificity among physiologically active substances is carried out, thereby detecting a substance associated with a living organism from sedimentation of gold fine particles or sequences.

In all of the above-described techniques, the surface where a physiologically active substance is immobilized is important. Surface plasmon resonance (SPR), which is most commonly used in this technical field, will be described below as an example.

A commonly used measurement chip comprises a transparent substrate (e.g., glass), an evaporated metal film, and a thin film having thereon a functional group capable of immobilizing a physiologically active substance. The measurement chip immobilizes the physiologically active substance on the metal surface via the functional group. A specific binding reaction between the physiological active substance and a test substance is measured, so as to analyze an interaction between biomolecules.

Regarding techniques for forming a metal thin film on a substrate material, for example, JP Patent Publication (Kokai) No. 60-86265 A (1985) discloses a technique that involves coating the large surface area of a float glass substrate material with a film-shaped metal thin film by a cathode sputtering method. JP Patent Publication (Kokai) No. 60-86265 A (1985) discloses a metal thin film that is made of a metal of gold, silver, copper, platinum, palladium or a mixture thereof showing low adhesive properties to a base material. To improve the adhesion of these metals to a substrate material surface, this technique involves adhering a primer layer such as stainless steel, steel, titanium, chromium, vanadium, aluminium or the like that binds to a substrate material surface under oxidation conditions and then sputtering the above metals having low adhesive property onto the primer layer.

Furthermore, JP Patent Publication (Kokai) No. 63-20073 A (1988) discloses a method that involves, for the film formation on a surface of the plastic lens substrate that is an optical part, pretreating the plastic substrate surface by plasma irradiation and then forming a film that can adhere well to plastic surfaces. This publication also discloses that impurities such as oil and water adsorbed to plastic surfaces are removed by phenomena such as impact desorption and impact decomposition that are induced by electrons and ions supplied from plasma, and that subsequently the plastic surface is activated by plasma particles.

### DISCLOSURE OF THE INVENTION

When a biosensor is produced by forming a metal thin film layer (up to 100 nm) on a resin surface, forming an extremely thin film of hydrophobic polymers on the thus formed film, and then chemically treating the hydrophobic polymer surfaces, a conventional technique causes plane deterioration (separation of a metal film from a resin) and results in a very poor yield. Thus, improvement of such a technique has been desired. Thus, an object of the present invention is to provide a biosensor which exhibits good reproducibility of the amount of an immobilized physiologically active substance by suppressing plane deterioration (separation of a metal film from a resin) which occurs at the formation of a thin metal film layer on a resin surface.

As a result of intensive studies to achieve the above object, the present inventors have discovered that a biosensor which exhibits good reproducibility of the amount of an immobilized physiologically active substance can be provided by performing plasma irradiation on a light-reflecting surface of an optical block made of a resin, forming a first metal layer, and then forming a second metal layer, so as to suppress plane deterioration (separation of a metal film from a resin) which occurs at the formation of metal layer. Further, the present inventors have discovered that a biosensor which exhibits good reproducibility of the amount of an immobilized physiologically active substance can be provided by forming metal layers on a light-reflecting surface of an optical block made of a resin with a high adhesion rate of metal atoms to the reflecting surface, so as to suppress plane deterioration (separation of a metal film from a resin) which occurs at the formation of a metal layer. The present invention has been completed based on these findings.

Thus, the present invention provides a biosensor which is produced by performing plasma irradiation on a light-reflecting surface of an optical block which is obtained by molding a resin and has at least 3 surfaces including a light incident surface, the light-reflecting surface and a light-emerging surface; forming a first metal layer; and then forming a second metal layer on the side of the first metal layer which is opposite to the light-reflecting surface.

Preferably, the resin is a synthetic resin, more preferably a nonpolar polymer, and particularly preferably a cyclic olefin polymer.

Preferably, plasma irradiation is performed by using a plasma discharge gas that is selected from Ar, N₂, or O₂.

Preferably, the light-reflecting surface of the optical block after plasma irradiation has surface roughness of Ra≦30 nm.

Preferably, the first metal layer and the second metal layer are composed of different metals, respectively.

Preferably, the first metal layer is composed of chromium, titanium, tantalum, nickel, or aluminium.

Preferably, the second metal layer is composed of gold or silver.

Preferably, the metal layer is formed by a sputtering method.

Preferably, the film thickness of the first metal layer is 5 nm or less.

Preferably, the film thickness of the second metal layer is between 20 nm and 100 nm.

Preferably, the particle size of the second metal layer is 30 nm or less.

Preferably, the first metal layer and/or the second metal layer is/are formed with an adhesion rate of metal atoms to the light-reflecting surface that is 10 nm/second or greater.

Preferably, the second metal layer is coated with a hydrophobic polymer layer.

Preferably, the hydrophobic polymer layer is surface-treated by chemical treatment.

Preferably, a block is pressed onto the light-reflecting surface so as to form a flow channel having the light-reflecting surface as a bottom face.

Preferably, the block which forms the flow channel is formed of a resin.

Preferably, the biosensor of the present invention is used for non-electrochemical detection and further more preferably used for surface plasmon resonance analysis.

In another aspect, the present invention provides a biosensor which is produced by forming a metal layer on a light-reflecting surface of an optical block which is obtained by molding a resin and has at least 3 surfaces including a light incident surface, the light-reflecting surface and a light-emerging surface, with an adhesion rate of metal atoms to the reflecting surface that is 10 nm/second or greater.

Preferably, the resin is a synthetic resin, more preferably a nonpolar polymer, and particularly preferably a cyclic olefin polymer.

Preferably, the metal layer is composed of gold or silver.

Preferably, the metal layer is formed by a sputtering method.

Preferably, the film thickness of the metal layer is between 20 nm and 100 nm.

Preferably, the metal layer is coated with a hydrophobic polymer layer.

Preferably, the hydrophobic polymer layer is surface-treated by chemical treatment.

Preferably, a block is pressed onto the light-reflecting surface so as to form a flow channel having the light-reflecting surface as a bottom face.

Preferably, the block which forms the flow channel is formed of a resin.

Preferably, the biosensor of the present invention is used for non-electrochemical detection and further more preferably used for surface plasmon resonance analysis.

In further another aspect, the present invention provides a method for producing the biosensor of the present invention, which comprises the steps of performing plasma irradiation on a light-reflecting surface of an optical block which is obtained by molding a resin and has at least 3 surfaces including a light incident surface, the light-reflecting surface and a light-emerging surface; forming a first metal layer on a plasma-irradiated light-irradiation surface; and then forming a second metal layer on the side of the first metal layer which is opposite to the light-reflecting surface.

In further another aspect, the present invention provides a method for producing a biosensor of the present invention, which comprises the step of forming a metal layer on a light-reflecting surface of an optical block which is obtained by molding a resin and has at least 3 surfaces including a light incident surface, the light-reflecting surface and a light-emerging surface, with an adhesion rate of metal atoms to the reflecting surface that is 10 nm/second or greater.

In further another aspect, the present invention provides the biosensor according to the present invention, wherein a physiologically active substance is bound to the surface.

In further another aspect, the present invention provides a method for immobilizing a physiologically active substance on a biosensor, which comprises a step of allowing a physiologically active substance to come into contact with the biosensor according to the present invention, so as to allow said physiologically active substance to bind to the surface of said biosensor.

In further another aspect, the present invention provides a method for detecting or measuring a substance interacting with a physiologically active substance, which comprises a step of allowing a test substance to come into contact with the biosensor according to the present invention to the surface of which the physiologically active substance binds via a covalent bond.

Preferably, the substance interacting with the physiologically active substance is detected or measured by a non-electrochemical method. More preferably, the substance interacting with the physiologically active substance is detected or measured by surface plasmon resonance analysis.

### BRIEF DESCRIPTION OF THE DRAWING

FIG. 1 shows a plastic prism used in the Examples.

FIG. 2 shows a position for the spin coating in the Examples.

FIG. 3 shows an examples of SPR systems for performing the screening according to the present invention.

### BEST MODE FOR CARRYING OUT THE INVENTION

The embodiments of the present invention will be described below.

According to the first embodiment, the biosensor of the present invention is characterized in that it is produced by performing plasma irradiation on a light-reflecting surface of an optical block which is obtained by molding a resin and has at least 3 surfaces including a light incident surface, the light-reflecting surface and a light-emerging surface; forming a first metal layer; and then forming a second metal layer on the side of the first metal layer which is opposite to the light-reflecting surface.

According to the second embodiment, the biosensor of the present invention is characterized in that it is produced by forming a metal layer on a light-reflecting surface of an optical block which is obtained by molding a resin and has at least 3 surfaces including a light incident surface, the light-reflecting surface and a light-emerging surface, with an adhesion rate of metal atoms to the reflecting surface that is 10 nm/second or greater.

The biosensor of the present invention has as broad a meaning as possible, and the term biosensor is used herein to mean a sensor, which converts an interaction between biomolecules into a signal such as an electric signal, so as to measure or detect a target substance. The conventional biosensor is comprised of a receptor site for recognizing a chemical substance as a detection target and a transducer site for converting a physical change or chemical change generated at the site into an electric signal. In a living body, there exist substances having an affinity with each other, such as enzyme/substrate, enzyme/coenzyme, antigen/antibody, or hormone/receptor. The biosensor operates on the principle that a substance having an affinity with another substance, as described above, is immobilized on a substrate to be used as a molecule-recognizing substance, so that the corresponding substance can be selectively measured.

Types of resin that composes an optical block are not specifically limited. Such resin may be either a synthetic resin or a natural resin and is preferably a synthetic resin. Such resin is preferably a nonpolar polymer. Specific examples of such resin include cyclic olefin polymer, polymethyl methacrylate, polyethyleneterephthalate, polycarbonate, and non-crystalline polyolefin.

Plasma irradiation can be performed using a plasma discharge gas that is selected from Ar, N₂, or O₂. For example, an optical block (prism) is installed in a substrate holder of a sputtering apparatus, the inside of which is caused to become a vacuum, Ar gas or the like is introduced, and then RF power (e.g., 0.5 kW) is applied to the substrate holder while rotating the substrate holder so that a prism surface can be plasma-treated. Furthermore, the light-reflecting surface of the optical block after plasma irradiation preferably has a surface roughness of Ra≦30 nm.

In the biosensor of the first embodiment of the present invention, the first metal layer and the second metal layer are preferably composed of different metals. Further, in the biosensor of the second embodiment of the present invention, a metal layer is formed on the optical block, and the metal layer may be a single layer, or two or more different layers may be provided. When two or more different layers may be provided, the first metal layer and the second metal layer are preferably composed of different metals.

Preferable examples of a metal composing the first metal layer include chromium, titanium, tantalum, nickel, and aluminium. A metal composing the second metal layer is not specifically limited as long as surface plasmon resonance can be generated, when such metals are used for a surface plasmon resonance biosensor. Preferable examples of such metal include free electron metals such as gold, silver, copper, aluminium, and platinum. In particular, a metal layer composed of gold or silver is preferable. The above metals can be used alone or in combination.

The first metal layer preferably has a film thickness of 5 nm or less. For example, a film thickness of between 1 nm and 5 nm can be employed. The second metal layer preferably has a film thickness of between 20 nm and 100 nm. Moreover, the second metal layer preferably has a particle size of 30 nm or less. For example, such a particle size of between 5 nm and 30 nm is further more preferable.

Metal layers may be formed by a standard method. For example, metal layers can be formed by a sputtering method, an evaporation method, an ion plating method, an electroplating method, or electroless plating. Particularly preferably, metal layers are formed by the sputtering method.

The sputtering method is one type of methods for forming a film on a substrate, and is generally performed by introducing an inert gas, such as Ar gas, into a vacuum. A substrate and a raw material (metal) for a film are provided close to each other, a vacuum is created, an inert gas is introduced, and then voltage is applied between the substrate and the raw material (metal) for the film. Electrons and ions then move at high speeds, and ions collide with the raw material (metal) for the film. The electrons and ions that have moved at high speeds collide with gas molecules so as to force out the electrons of the molecules and result in ions. The ions that have collided with the raw material (metal) for the film force out particles of the raw material (metal) for the film (this is also referred to as a sputtering phenomenon). The forced out particles (metal) collide with and become adhered to the substrate, so that the film is formed.

Specific examples of a sputtering apparatus for performing the sputtering method are as follows.

(1) Double-Pole Dc Glow Discharge Sputtering Apparatus

When several hundred volts are applied between a substrate and a target (in the present invention, metal) in an inert gas (e.g., Ar) at approximately 1 to 10⁻² Torr, ionized Ar collides with the target at accelerated speed, and then the target substance is sputtered and deposited on the substrate. Simultaneously, high-energy γ electrons are generated from the target, and then collide with Ar atoms so as to ionize the Ar atoms, thereby maintaining plasma.

(2) Magnetron Sputtering Apparatus

A magnetron sputtering apparatus is characterized by placing a magnet on the back side of a target (metal) and then applying a magnetic field to enclose γ electrons in the vicinity of the target. The γ electrons achieve an orbit sticking to the line of magnetic force so that plasma is centered in the vicinity of the target and damage to the substrate can be reduced. Also, the movement distances of the γ electrons become longer, so that high-speed sputtering becomes possible with low gas pressure.

(3) Ion Beam Sputtering Method

A film deposition chamber is separated from an ion-generating chamber. A target is irradiated with ions released from a plasma ion chamber, thereby causing sputtering. With this apparatus, plasma does not invade the deposition chamber. Hence, no adverse effects are caused by γ electrons or the like and the energy and the density of ions that are transmitted toward the target can be independently controlled.

In the biosensor of the first embodiment of the present invention, it is preferable to form the first metal layer and/or the second metal layer with an adhesion rate of metal atoms to the light-reflecting surface that is 10 nm/second or greater.

In the biosensor of the second embodiment of the present invention, metal layer is formed on the light-reflecting surface with an adhesion rate of metal atoms to the reflecting surface that is 10 nm/second or greater. Such adhesion rate of metal atoms to the reflecting surface is preferably 10 nm/second or greater and 200 nm/second or less, further more preferably 10 nm/second or greater and 100 nm/second or less, and particularly preferably 10 nm/second or greater and 50 nm/second or less.

For example, when metal layer is formed by the sputtering method, the adhesion rate of metal atoms to the reflecting surface can be controlled by controlling the voltage to be applied.

In the present invention, a block can be pressed onto the light-reflecting surface so as to form a flow channel having the light-reflecting surface as a bottom face. As a block for the formation of the flow channel, for example, a block formed of a resin can be used.

Preferably in the present invention, the second metal layer is coated with a hydrophobic polymer layer. A hydrophobic polymer used in the present invention is a polymer having no water-absorbing properties. Its solubility in water (25° C.) is 10% or less, more preferably 1% or less, and most preferably 0.1% or less.

A hydrophobic monomer which forms a hydrophobic polymer can be selected from vinyl esters, acrylic esters, methacrylic esters, olefins, styrenes, crotonic esters, itaconic diesters, maleic diesters, fumaric diesters, allyl compounds, vinyl ethers, vinyl ketones, or the like. The hydrophobic polymer may be either a homopolymer consisting of one type of monomer, or copolymer consisting of two or more types of monomers.

Examples of a hydrophobic polymer that is preferably used in the present invention may include polystyrene, polyethylene, polypropylene, polyethylene terephthalate, polyvinyl chloride, polymethyl methacrylate, polyester, and nylon.

In the present invention, a hydrophobic polymer having an alkyl group substituted with a fluorine atom can be used. A hydrophobic monomer which forms a hydrophobic polymer having an alkyl group substituted with a fluorine atom can be selected from vinyl esters, acrylic esters, methacrylic esters, olefins, styrenes, crotonic esters, itaconic diesters, maleic diesters, fumaric diesters, allyl compounds, vinyl ethers, vinyl ketones, or the like. The hydrophobic polymer may be either a homopolymer consisting of one type of monomer, or copolymer consisting of two or more types of monomers.

Such a hydrophobic polymer having an alkyl group substituted with a fluorine atom preferably has a fluorinated alkyl group as an ester in a molecule thereof. In particular, acrylic ester and methacrylic ester are preferable.

Such a fluorinated alkyl group may be a linear, branched, or cyclic group containing 1 or more carbon atoms. (Hereinafter, an alkyl group substituted with a fluorine atom is referred to as “Rf.”)

Rf is an alkyl group containing 1 or more carbon atoms, which is substituted with at least one fluorine atom. Rf may be substituted with at least one fluorine atom, and may have a linear, branched, or cyclic structure. In addition, such Rf may further be substituted with substituents other than a fluorine atom, or it may be substituted only with a fluorine atom.

Examples of substituents other than a fluorine atom for Rf may include an alkenyl group, an aryl group, an alkoxyl group, halogen atoms other than a fluorine atom, a carboxylic ester group, a carbonamide group, a carbamoyl group, an oxycarbonyl group, and a phosphoric ester group.

As Rf, a fluorine-substituted alkyl group containing 1 to 16 carbon atoms is preferable, a fluorine-substituted alkyl group containing 1 to 12 carbon atoms is more preferable, and a fluorine-substituted alkyl group containing 4 to 10 carbon atoms is further more preferable. Preferred examples of such Rf are given below.

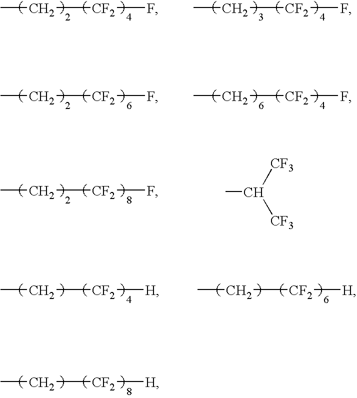

Rf is further preferably an alkyl group containing 4 to 10 carbon atoms, the terminus of which is substituted with a trifluoromethyl group, and is particularly preferably an alkyl group containing 3 to 10 carbon atoms which is represented by —(CH₂)ₙ₁—(CF₂)ₙ₂F wherein n¹ represents an integer between 1 and 6, and n² represents an integer between 3 and 8. Specific examples of Rf may include —CH₂—(CF₂)₂F, —(CH₂)₆—(CF₂)₄F, —(CH₂)₃—(CF₂)₄F, —CH₂—(CF₂)₃F, —(CH₂)₂—(CF₂)₄F, —(CH₂)₃—(CF₂)₄F, —(CH₂)₆—(CF₂)₄F, —(CH₂)₂—(CF₂)₆F, —(CH₂)₃—(CF₂)₆F, and —(CH₂)₂—(CF₂)₆F. Of these, —(CH₂)₂—(CF₂)₄F and —(CH₂)₂—(CF₂)₆F are most preferable.

A hydrophobic polymer having an alkyl group substituted with a fluorine atom used in the present invention may also be a copolymer with other monomers. In such a case, preferred examples of a copolymer may include methacrylic esters such as methyl methacrylate, acrylic esters such as methyl acrylate, and styrene.

Specific examples of a hydrophobic polymer having an alkyl group substituted with a fluorine atom are given below.

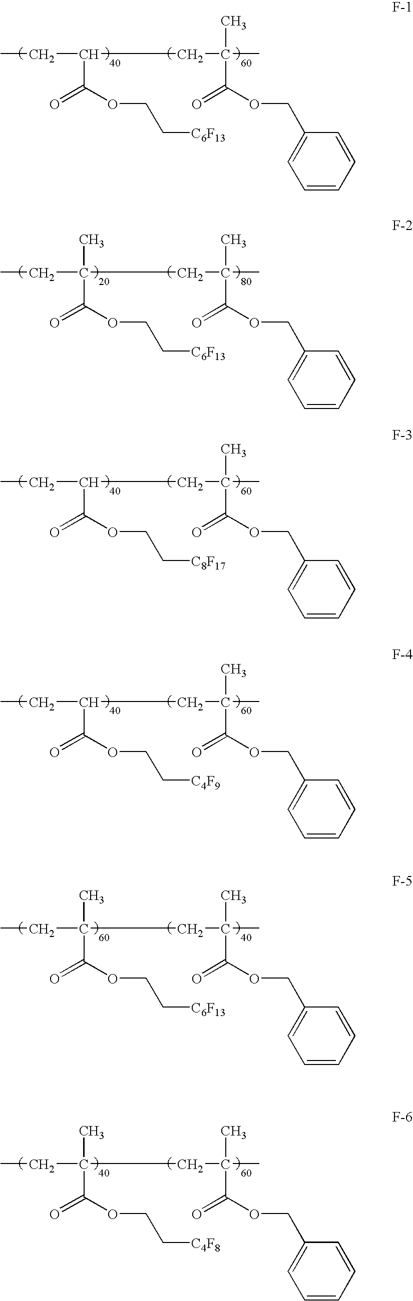

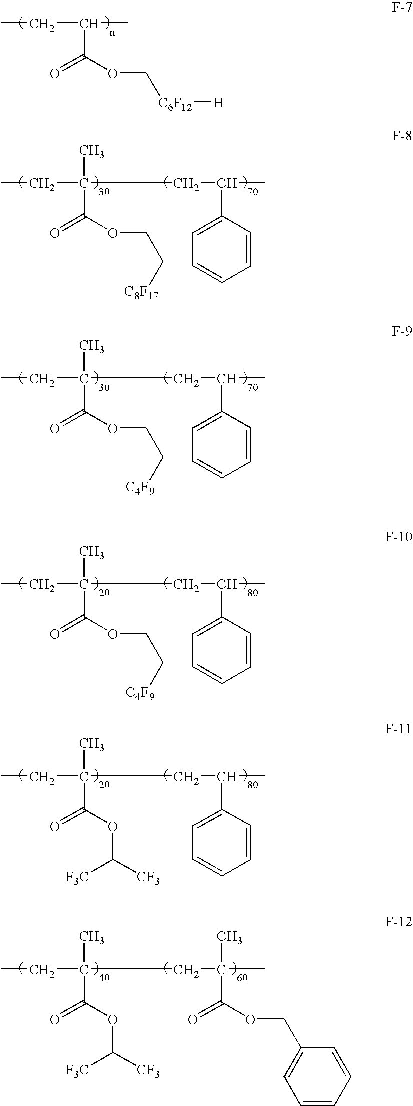

A metal layer is coated with a hydrophobic polymer according to common methods. Examples of such a coating method may include spin coating, air knife coating, bar coating, blade coating, slide coating, curtain coating, spray method, evaporation method, cast method, and dip method. Among them, spin coating and dip method are mentioned in detail below.

Spin-coating is a method for producing a thin film by adding a solution dropwise to a substrate placed on a rotating disk, wherein the thickness of a film is controlled by the concentration of the solution, the number of rotations of the disk, the vapor pressure of a solvent, etc.

The concentration of a hydrophobic polymer contained in a solution used in the spin-coating is preferably between 0.001% by weight and 50% by weight, more preferably between 0.01% by weight and 10% by weight, and further preferably between 0.1% by weight and 5% by weight.

The number of rotations of the disk during spin coating is preferably between 10 rpm and 10,000 rpm, more preferably between 50 rpm and 7,500 rpm, and further preferably between 100 rpm and 5,000 rpm.

A solvent used in the spin-coating has a vapor pressure preferably between 0.1 kPa and 100 kPa, more preferably between 0.5 kPa and 50 kPa, and further preferably between 1 kPa and 30 kPa, at the environmental temperature applied during the production of a thin film by spin coating. Specific examples of a solvent used herein may include methanol, ethanol, i-propanol, n-butanol, t-butanol, acetone, methyl ethyl ketone, methyl isobutyl ketone, ethyl acetate, butyl acetate, dimethyl sulfoxide, and dimethyl formamide.

Preferably in the present invention, a coating solution is added dropwise to the surface of a substrate (namely, an optical block on which a first and second metal layers are formed) retained on a disk, and the disk is rotated.

When the coating solution is added dropwise to the substrate surface, the coating rate on the substrate surface with the coating solution is preferably between 80% and 100%, and preferably between 90% and 100%.

In the dip method, coating is carried out by contacting a substrate with a solution of a hydrophobic polymer, and then with a liquid which does not contain the hydrophobic polymer. Preferably, the solvent of the solution of a hydrophobic polymer is the same as that of the liquid which does not contain said hydrophobic polymer.

In the dip method, a layer of a hydrophobic polymer having an uniform coating thickness can be obtained on a surface of a substrate regardless of inequalities, curvature and shape of the substrate by suitably selecting a coating solvent for hydrophobic polymer.

The type of coating solvent used in the dip method is not particularly limited, and any solvent can be used so long as it can dissolve a part of a hydrophobic polymer. Examples thereof include formamide solvents such as N,N-dimethylformamide, nitrile solvents such as acetonitrile, alcohol solvents such as phenoxyethanol, ketone solvents such as 2-butanone, and benzene solvents such as toluene, but are not limited thereto.

In the solution of a hydrophobic polymer which is contacted with a substrate, the hydrophobic polymer may be dissolved completely, or alternatively, the solution may be a suspension which contains undissolved component of the hydrophobic polymer. The temperature of the solution is not particularly limited, so long as the state of the solution allows a part of the hydrophobic polymer to be dissolved. The temperature is preferably −20° C. to 100° C. The temperature of the solution may be changed during the period when the substrate is contacted with a solution of a hydrophobic polymer. The concentration of the hydrophobic polymer in the solution is not particularly limited, and is preferably 0.01% to 30%, and more preferably 0.1% to 10%.

The period for contacting the solid substrate with a solution of a hydrophobic polymer is not particularly limited, and is preferably 1 second to 24 hours, and more preferably 3 seconds to 1 hour.

As the liquid which does not contain the hydrophobic polymer, it is preferred that the difference between the SP value (unit: (J/cm³)^{1/2}) of the solvent itself and the SP value of the hydrophobic polymer is 1 to 20, and more preferably 3 to 15. The SP value is represented by a square root of intermolecular cohesive energy density, and is referred to as solubility parameter. In the present invention, the SP value δ was calculated by the following formula. As the cohesive energy (Ecoh) of each functional group and the mol volume (V), those defined by Fedors were used (R. F. Fedors, Polym. Eng. Sci., 14(2), P147, P472(1974)).
Δ=(ΣEcoh/ΣV)^{1/2}

Examples of the SP values of the hydrophobic polymers and the solvents are shown below;

Solvent: 2-phenoxyethanol: 25.3 against polymethylmethacrylate-polystyrene:copolymer (1:1): 21.0

Solvent: acetonitrile: 22.9 against polymethylmethacrylate: 20.3

Solvent: toluene: 18.7 against polystyrene: 21.6

The period for contacting a substrate with a liquid which does not contain the hydrophobic polymer is not particularly limited, and is preferably 1 second to 24 hours, and more preferably 3 seconds to 1 hour. The temperature of the liquid is not particularly limited, so long as the solvent is in a liquid state, and is preferably −20° C. to 100° C. The temperature of the liquid may be changed during the period when the substrate is contacted with the solvent. When a less volatile solvent is used, the less volatile solvent may be substituted with a volatile solvent which can be dissolved in each other after the substrate is contacted with the less volatile solvent, for the purpose of removing the less volatile solvent.

The coating thickness of a hydrophobic polymer is not particularly limited, but it is preferably between 0.1 nm and 500 nm, and particularly preferably between 1 nm and 300 nm.

The biosensor composed of a substrate coated with a hydrophobic polymer preferably has a functional group capable of immobilizing a physiologically active substance on the outermost surface of the substrate. The term “the outermost surface of the substrate” is used to mean “the surface, which is farthest from the substrate,” and more specifically, it means “the surface of a hydrophobic polymer applied on a substrate, which is farthest from the substrate.”

Preferred functional group includes —OH, —SH, —COOH, —NR¹R² (wherein each of R¹ and R² independently represents a hydrogen atom or lower alkyl group), —CHO, —NR³NR¹R² (wherein each of R¹, R² and R³ independently represents a hydrogen atom or lower alkyl group), —NCO, —NCS, an epoxy group, or a vinyl group. The number of carbon atoms contained in the lower alkyl group is not particularly limited herein. However, it is generally about C1 to C10, and preferably C1 to C6.

In order to introduce these functional groups into the surface, a method is applied that involves applying a hydrophobic polymer containing a precursor of such a functional group on a metal surface or metal film, and then generating the functional group from the precursor located on the outermost surface by chemical treatment. For example, polymethyl methacrylate, a hydrophobic polymer containing —COOCH₃ group, is applied on a metal film, and then the surface comes into contact with an NaOH aqueous solution (1N) at 40° C. for 16 hours, so that a —COOH group is generated on the outermost surface.

A physiologically active substance is covalently bound to the above-obtained surface for a biosensor via the above functional group, so that the physiologically active substance can be immobilized on the metal surface or metal film.

A physiologically active substance immobilized on the surface for the biosensor of the present invention is not particularly limited, as long as it interacts with a measurement target. Examples of such a substance may include an immune protein, an enzyme, a microorganism, nucleic acid, a low molecular weight organic compound, a nonimmune protein, an immunoglobulin-binding protein, a sugar-binding protein, a sugar chain recognizing sugar, fatty acid or fatty acid ester, and polypeptide or oligopeptide having a ligand-binding ability.

Examples of an immune protein may include an antibody whose antigen is a measurement target, and a hapten. Examples of such an antibody may include various immunoglobulins such as IgG, IgM, IgA, IgE or IgD. More specifically, when a measurement target is human serum albumin, an anti-human serum albumin antibody can be used as an antibody. When an antigen is an agricultural chemical, pesticide, methicillin-resistant Staphylococcus aureus, antibiotic, narcotic drug, cocaine, heroin, crack or the like, there can be used, for example, an anti-atrazine antibody, anti-kanamycin antibody, anti-metamphetamine antibody, or antibodies against O antigens 26, 86, 55, 111 and 157 among enteropathogenic Escherichia coli.

An enzyme used as a physiologically active substance herein is not particularly limited, as long as it exhibits an activity to a measurement target or substance metabolized from the measurement target. Various enzymes such as oxidoreductase, hydrolase, isomerase, lyase or synthetase can be used. More specifically, when a measurement target is glucose, glucose oxidase is used, and when a measurement target is cholesterol, cholesterol oxidase is used. Moreover, when a measurement target is an agricultural chemical, pesticide, methicillin-resistant Staphylococcus aureus, antibiotic, narcotic drug, cocaine, heroin, crack or the like, enzymes such as acetylcholine esterase, catecholamine esterase, noradrenalin esterase or dopamine esterase, which show a specific reaction with a substance metabolized from the above measurement target, can be used.

A microorganism used as a physiologically active substance herein is not particularly limited, and various microorganisms such as Escherichia coli can be used.

As nucleic acid, those complementarily hybridizing with nucleic acid as a measurement target can be used. Either DNA (including cDNA) or RNA can be used as nucleic acid. The type of DNA is not particularly limited, and any of native DNA, recombinant DNA produced by gene recombination and chemically synthesized DNA may be used.

As a low molecular weight organic compound, any given compound that can be synthesized by a common method of synthesizing an organic compound can be used.

A nonimmune protein used herein is not particularly limited, and examples of such a nonimmune protein may include avidin (streptoavidin), biotin, and a receptor.

Examples of an immunoglobulin-binding protein used herein may include protein A, protein G, and a rheumatoid factor (RF).

As a sugar-binding protein, for example, lectin is used.

Examples of fatty acid or fatty acid ester may include stearic acid, arachidic acid, behenic acid, ethyl stearate, ethyl arachidate, and ethyl behenate.

A biosensor to which a physiologically active substance is immobilized as described above can be used to detect and/or measure a substance which interacts with the physiologically active substance.

Thus, the present invention provides a method of detecting and/or measuring a substance interacting with the physiologically active substance immobilized to the biosensor of the present invention, to which a physiologically active substance is immobilized, wherein the biosensor is contacted with a test substance.

As such a test substance, for example, a sample containing the above substance interacting with the physiologically active substance can be used.

In the present invention, it is preferable to detect and/or measure an interaction between a physiologically active substance immobilized on the surface used for a biosensor and a test substance by a nonelectric chemical method. Examples of a non-electrochemical method may include a surface plasmon resonance (SPR) measurement technique, a quartz crystal microbalance (QCM) measurement technique, and a measurement technique that uses functional surfaces ranging from gold colloid particles to ultra-fine particles.

In a preferred embodiment of the present invention, the biosensor of the present invention can be used as a biosensor for surface plasmon resonance.

A biosensor for surface plasmon resonance is a biosensor used for a surface plasmon resonance biosensor, meaning a member comprising a portion for transmitting and reflecting light emitted from the sensor and a portion for immobilizing a physiologically active substance. It may be fixed to the main body of the sensor or may be detachable.

The surface plasmon resonance phenomenon occurs due to the fact that the intensity of monochromatic light reflected from the border between an optically transparent substance such as glass and a metal thin film layer depends on the refractive index of a sample located on the outgoing side of the metal. Accordingly, the sample can be analyzed by measuring the intensity of reflected monochromatic light.

A device using a system known as the Kretschmann configuration is an example of a surface plasmon measurement device for analyzing the properties of a substance to be measured using a phenomenon whereby a surface plasmon is excited with a lightwave (for example, Japanese Patent Laid-Open No. 6-167443). The surface plasmon measurement device using the above system basically comprises a dielectric block formed in a prism state, a metal film that is formed on a face of the dielectric block and comes into contact with a measured substance such as a sample solution, a light source for generating a light beam, an optical system for allowing the above light beam to enter the dielectric block at various angles so that total reflection conditions can be obtained at the interface between the dielectric block and the metal film, and a light-detecting means for detecting the state of surface plasmon resonance, that is, the state of attenuated total reflection, by measuring the intensity of the light beam totally reflected at the above interface.

In order to achieve various incident angles as described above, a relatively thin light beam may be caused to enter the above interface while changing an incident angle. Otherwise, a relatively thick light beam may be caused to enter the above interface in a state of convergent light or divergent light, so that the light beam contains components that have entered therein at various angles. In the former case, the light beam whose reflection angle changes depending on the change of the incident angle of the entered light beam can be detected with a small photodetector moving in synchronization with the change of the above reflection angle, or it can also be detected with an area sensor extending along the direction in which the reflection angle is changed. In the latter case, the light beam can be detected with an area sensor extending to a direction capable of receiving all the light beams reflected at various reflection angles.

With regard to a surface plasmon measurement device with the above structure, if a light beam is allowed to enter the metal film at a specific incident angle greater than or equal to a total reflection angle, then an evanescent wave having an electric distribution appears in a measured substance that is in contact with the metal film, and a surface plasmon is excited by this evanescent wave at the interface between the metal film and the measured substance. When the wave vector of the evanescent light is the same as that of a surface plasmon and thus their wave numbers match, they are in a resonance state, and light energy transfers to the surface plasmon. Accordingly, the intensity of totally reflected light is sharply decreased at the interface between the dielectric block and the metal film. This decrease in light intensity is generally detected as a dark line by the above light-detecting means. The above resonance takes place only when the incident beam is p-polarized light. Accordingly, it is necessary to set the light beam in advance such that it enters as p-polarized light.

If the wave number of a surface plasmon is determined from an incident angle causing the attenuated total reflection (ATR), that is, an attenuated total reflection angle (θSP), the dielectric constant of a measured substance can be determined. As described in Japanese Patent Laid-Open No. 11-326194, a light-detecting means in the form of an array is considered to be used for the above type of surface plasmon measurement device in order to measure the attenuated total reflection angle (θSP) with high precision and in a large dynamic range. This light-detecting means comprises multiple photo acceptance units that are arranged in a certain direction, that is, a direction in which different photo acceptance units receive the components of light beams that are totally reflected at various reflection angles at the above interface.

In the above case, there is established a differentiating means for differentiating a photodetection signal outputted from each photo acceptance unit in the above array-form light-detecting means with regard to the direction in which the photo acceptance unit is arranged. An attenuated total reflection angle (θSP) is then specified based on the derivative value outputted from the differentiating means, so that properties associated with the refractive index of a measured substance are determined in many cases.

In addition, a leaking mode measurement device described in “Bunko Kenkyu (Spectral Studies)” Vol. 47, No. 1 (1998), pp. 21 to 23 and 26 to 27 has also been known as an example of measurement devices similar to the above-described device using attenuated total reflection (ATR). This leaking mode measurement device basically comprises a dielectric block formed in a prism state, a clad layer that is formed on a face of the dielectric block, a light wave guide layer that is formed on the clad layer and comes into contact with a sample solution, a light source for generating a light beam, an optical system for allowing the above light beam to enter the dielectric block at various angles so that total reflection conditions can be obtained at the interface between the dielectric block and the clad layer, and a light-detecting means for detecting the excitation state of waveguide mode, that is, the state of attenuated total reflection, by measuring the intensity of the light beam totally reflected at the above interface.

In the leaking mode measurement device with the above structure, if a light beam is caused to enter the clad layer via the dielectric block at an incident angle greater than or equal to a total reflection angle, only light having a specific wave number that has entered at a specific incident angle is transmitted in a waveguide mode into the light wave guide layer, after the light beam has penetrated the clad layer. Thus, when the waveguide mode is excited, almost all forms of incident light are taken into the light wave guide layer, and thereby the state of attenuated total reflection occurs, in which the intensity of the totally reflected light is sharply decreased at the above interface. Since the wave number of a waveguide light depends on the refractive index of a measured substance placed on the light wave guide layer, the refractive index of the measurement substance or the properties of the measured substance associated therewith can be analyzed by determining the above specific incident angle causing the attenuated total reflection.

In this leaking mode measurement device also, the above-described array-form light-detecting means can be used to detect the position of a dark line generated in a reflected light due to attenuated total reflection. In addition, the above-described differentiating means can also be applied in combination with the above means.

The above-described surface plasmon measurement device or leaking mode measurement device may be used in random screening to discover a specific substance binding to a desired sensing substance in the field of research for development of new drugs or the like. In this case, a sensing substance is immobilized as the above-described measured substance on the above thin film layer (which is a metal film in the case of a surface plasmon measurement device, and is a clad layer and a light guide wave layer in the case of a leaking mode measurement device), and a sample solution obtained by dissolving various types of test substance in a solvent is added to the sensing substance. Thereafter, the above-described attenuated total reflection angle (θSP) is measured periodically when a certain period of time has elapsed.

If the test substance contained in the sample solution is bound to the sensing substance, the refractive index of the sensing substance is changed by this binding over time. Accordingly, the above attenuated total reflection angle (θSP) is measured periodically after the elapse of a certain time, and it is determined whether or not a change has occurred in the above attenuated total reflection angle (θSP), so that a binding state between the test substance and the sensing substance is measured. Based on the results, it can be determined whether or not the test substance is a specific substance binding to the sensing substance. Examples of such a combination between a specific substance and a sensing substance may include an antigen and an antibody, and an antibody and an antibody. More specifically, a rabbit anti-human IgG antibody is immobilized as a sensing substance on the surface of a thin film layer, and a human IgG antibody is used as a specific substance.

It is to be noted that in order to measure a binding state between a test substance and a sensing substance, it is not always necessary to detect the angle itself of an attenuated total reflection angle (θSP). For example, a sample solution may be added to a sensing substance, and the amount of an attenuated total reflection angle (θSP) changed thereby may be measured, so that the binding state can be measured based on the magnitude by which the angle has changed. When the above-described array-form light-detecting means and differentiating means are applied to a measurement device using attenuated total reflection, the amount by which a derivative value has changed reflects the amount by which the attenuated total reflection angle (θSP) has changed. Accordingly, based on the amount by which the derivative value has changed, a binding state between a sensing substance and a test substance can be measured (Japanese Patent Application No. 2000-398309 filed by the present applicant). In a measuring method and a measurement device using such attenuated total reflection, a sample solution consisting of a solvent and a test substance is added dropwise to a cup- or petri dish-shaped measurement chip wherein a sensing substance is immobilized on a thin film layer previously formed at the bottom, and then, the above-described amount by which an attenuated total reflection angle (θSP) has changed is measured.

Moreover, Japanese Patent Laid-Open No. 2001-330560 describes a measurement device using attenuated total reflection, which involves successively measuring multiple measurement chips mounted on a turntable or the like, so as to measure many samples in a short time.

When the biosensor of the present invention is used in surface plasmon resonance analysis, it can be applied as a part of various surface plasmon measurement devices described above.

The present invention will be further specifically described in the following examples. However, the examples are not intended to limit the scope of the present invention.

### EXAMPLES

### Example X1

The sensor chip of the present invention was produced by the following method.

(1) Deposition of Gold Film onto Plastic Prism

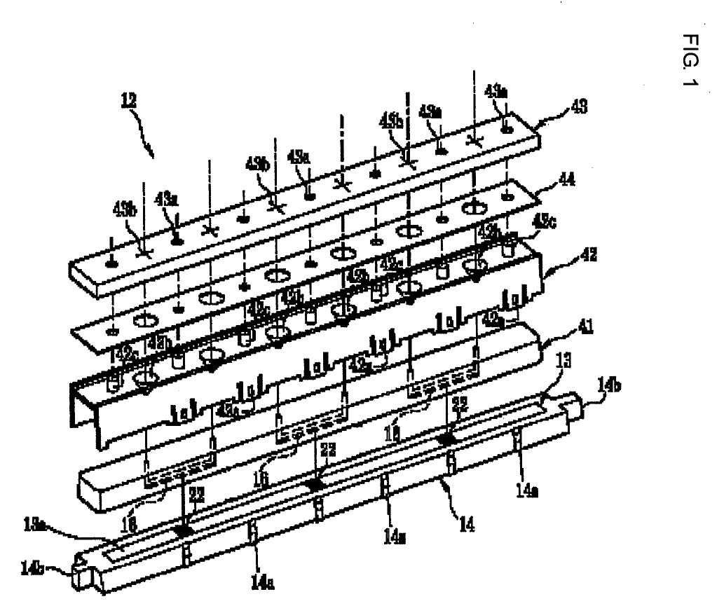

A gold thin film was deposited on the upper surface of a plastic prism (FIG. 1) obtained by injection molding of ZEONEX (produced by ZEON CORPORATION) by the following method.

(1-1) Deposition of Gold Film

A prism was installed in a substrate holder of a sputtering apparatus, the inside of which was caused to become a vacuum (base pressure 1×10⁻³ Pa or less), and then Ar gas was introduced therein (1 Pa). RF power (0.5 kW) was applied to the substrate holder for approximately 9 minutes while rotating (20 rpm) the substrate holder, thereby performing plasma treatment (also referred to as substrate etching or reverse sputtering) for the prism surface. After plasma irradiation, the light-reflecting surface of an optical block had surface roughness of Ra≦30 nm. Next, the introduction of Ar gas was stopped, and then the inside of the apparatus was caused to become a vacuum. Ar gas was introduced again (0.5 Pa), and then DC power (0.2 kW) was applied to an 8-inch Cr target for approximately 30 seconds while rotating (10 rpm to 40 rpm) the substrate holder, thereby forming a 2-nm Cr thin film. Next, introduction of Ar gas was stopped, and then the inside of the apparatus was caused to become a vacuum again. Ar gas was introduced again (0.5 Pa) and then DC power (1 kW) was applied to an 8-inch Au target for approximately 50 seconds while rotating (20 rpm) the substrate holder, thereby forming an Au thin film of approximately 50 nm. The particle size of Au was approximately 20 nm. The thus obtained samples are referred to as chip XA.

(2) Polymer Coating

Polymer thin film was deposited on the gold thin films of chip XA by the following method.

(2-1) Preparation of Polymer Solution A

1.5 g of polymer (F-1) was dissolved in 100 mL of anhydrous MiBK (methyl isobutyl ketone), and then the resultant was filtered using a microfilter with a pore size of 0.45 μm. The moisture content of anhydrous MiBK was 20 ppm. 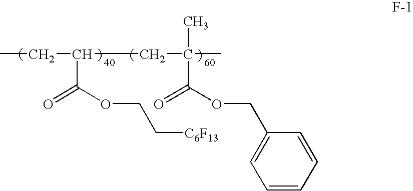
(2-2) Spin Coating

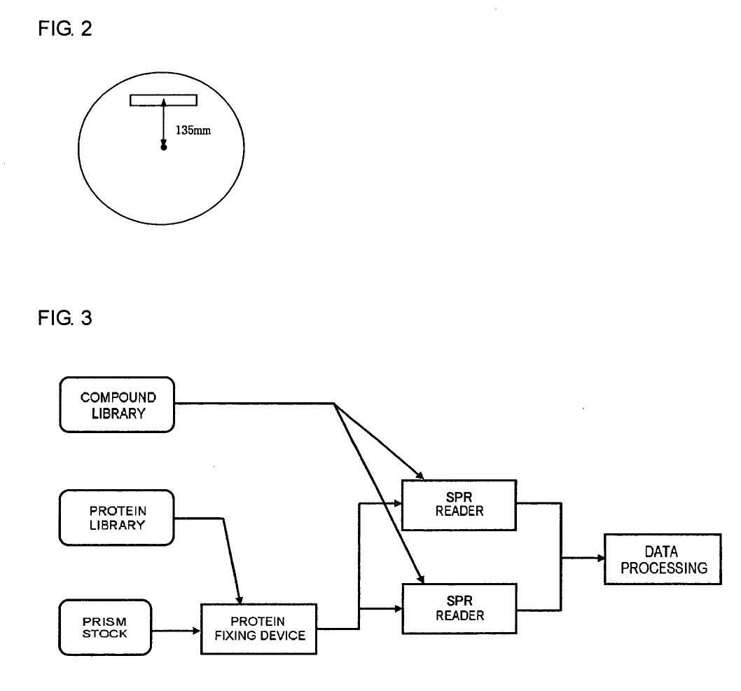

The chip XA was set in a spin coater (SC-408S sample seal-type spin coater, produced by a private limited company, Oshigane). The chip XA was fixed at a position 135 mm away from the center of the spin coater as shown in FIG. 2. 200 μL of a polymer solution A was casted so as to cover the entire gold film surface of the chip XA. Next, a wind-shielding cover was set so as to completely cover the chip XA, followed by spinning at 200 rpm for 60 seconds. After rotation was stopped, the chip XA was allowed to stand for 5 minutes.

(2-3) Vacuum Drying

The chip XA spin-coated with the polymer was vacuum-dried for 16 hours. The thus obtained samples are referred to as chip XB.

(3) Hydrolysis of Polymer Surface

The polymer thin film surface of the chip XB was hydrolyzed and then COOH groups were generated on the upper most surface by the following method.

(3-1) Hydrolysis

The chip XB was immersed in 1 N NaOH solution, followed by 16 hours of storage in a thermostatic bath at 60° C.

(3-2) Washing

After being extracted from the thermostatic bath at 60° C., the chip XB was naturally cooled for 15 minutes and then washed with extra pure water. The thus obtained samples are referred to as chips XC.

### Comparative Example X1

### Plasma Treatment→Gold Film

The following method was performed instead of the method in Example X1 (1), “Deposition of gold film onto plastic prism”. The other processes were performed in a manner similar to that in Example X1. Thus, a comparative sensor chip was produced. The thus obtained sample is referred to as chip XD.

(1) Deposition of Gold Film onto Plastic Prism

A gold thin film was deposited on the upper surface of a plastic prism (FIG. 1) obtained by injection molding of ZEONEX (produced by ZEON CORPORATION) by the following method.

(1-1) Deposition of Gold Film

A prism was installed in a substrate holder of a sputtering apparatus, the inside of which was caused to become a vacuum (base pressure 1×10⁻³ Pa or less), and then Ar gas was introduced therein (1 Pa). RF power (0.5 kW) was applied to the substrate holder for approximately 9 minutes while rotating (20 rpm) the substrate holder, thereby performing plasma treatment (also referred to as substrate etching or reverse sputtering) for the prism surface. Next, the introduction of Ar gas was stopped, and then the inside of the apparatus was again caused to become a vacuum. Ar gas was introduced again (0.5 Pa), and then DC power (1 kW) was applied to an 8-inch Au target for approximately 50 seconds while rotating (20 rpm) the substrate holder, thereby forming an Au thin film of approximately 50 nm.

### Comparative Example X2

### No Plasma Treatment→Cr Film→Gold Film

The following method was performed instead of the method in Example X1 (1), “Deposition of gold film onto plastic prism”. The other processes were performed in a manner similar to that in Example X1. Thus, a comparative sensor chip was produced. The thus obtained sample is referred to as chips XE.

(1) Deposition of Gold Film onto Plastic Prism

A gold thin film was deposited on the upper surface of a plastic prism (FIG. 1) obtained by injection molding of ZEONEX (produced by ZEON CORPORATION) by the following method.

(1-1) Deposition of Gold Film

A prism was installed in a substrate holder of a sputtering apparatus, the inside of which was caused to become a vacuum (base pressure 1×10⁻³ Pa or less), and then Ar gas was introduced therein (0.5 Pa). DC power (0.2 kW) was applied to an 8-inch Cr target for approximately 30 seconds while rotating (10 rpm to 40 rpm) the substrate holder, thereby forming a 2-nm Cr thin film. Next, the introduction of Ar gas was stopped, and then the inside of the apparatus was again caused to become a vacuum. Ar gas was introduced again (0.5 Pa) and then DC power (1 kW) was applied to an 8-inch Au target for approximately 50 seconds while rotating (20 rpm) the substrate holder, thereby forming an Au thin film of approximately 50 nm.

### Comparative Example X3

### Gold Film Only

The following method was performed instead of the method in Example X1 (1), “Deposition of gold film onto plastic prism”. The other processes were performed in a manner similar to that in Example X1. Thus, a comparative sensor chip was produced. The thus obtained sample is referred to as chips XF.

(1) Deposition of Gold Film onto Plastic Prism

A gold thin film was deposited on the upper surface of a plastic prism (FIG. 1) obtained by injection molding of ZEONEX (produced by ZEON CORPORATION) by the following method.

(1-1) Deposition of Gold Film

A prism was installed in a substrate holder of a sputtering apparatus, the inside of which was caused to become a vacuum (base pressure 1×10⁻³ Pa or less), and then Ar gas was introduced therein (0.5 Pa). DC power (1 kW) was applied to an 8-inch Au target for approximately 50 seconds while rotating (20 rpm) the substrate holder, thereby forming an Au thin film of approximately 50 nm.

### Test Example X1

### Determination of the Number of Separation

The number of separations was determined using the chips XC (of the present invention) and the chips XD, XE, and XF (of comparative examples). Determination was performed by measuring the number of separation (cylindrically generated) in each of 5 visual fields using a ×20 light microscope and obtaining average numbers thereof. Table 1 shows the results. Only in the case of the chip XC of the present invention, no separation was generated.

TABLE 1

| | Average number of | |
|---|---|---|
| | separation | Remarks |
| Chip XC | 0 | Invention |
| Chip XD | Complete separation of | Comparative |
| | gold film | |
| Chip XE | Complete separation of | Comparative |
| | gold film | |
| Chip CF | 50 | Comparative |

### Test Example X2

### Surface Treatment of Chips

The surfaces of chips XC and XF were treated by the following method.

(1) 5-aminovaleric Acid Binding

5-aminovaleric acid was covalently bound to COOH groups existing on the chip surfaces by the following method.

(1-1) Preparation of Activation Solutions and 5-aminovaleric Acid Solution

0.1 M NHS solution: 1.16 g of NHS(N-hydroxysuccinic acid) was dissolved in extra pure water to a volume of 100 mL.

0.4 M EDC solution: 7.7 g of EDC (hydrochloric acid 1-ethyl-3-(3-dimethylaminopropyl) carbodiimide) was dissolved in extra pure water to a volume of 100 mL.

1 M 5-aminovaleric acid solution: 11.7 g of 5-aminovaleric acid was dissolved in 80 mL of extra pure water, and then the resultant was adjusted to pH 8.5 using 1N NaOH. Extra pure water was further added to the resultant to a volume of 100 mL.

(1-2) Activation

A chip was dried well using an air gun. The chip was then set in a damp box (a tight box wherein a wet cloth had been placed, and humidity of 90% RH or more was maintained under sealed conditions). 200 μL of a mixed solution of 100 μL of 0.1 M NHS solution and 100 μL of 0.4 M EDC solution was casted. A PET film having a thickness of 175 μm which was cut to have a size of 120 mm×8.5 mm was placed on the solution, so as to cover the surface while spreading the solution. The surface area ratio of the portion of the solution that was not in contact with air to the portion that was in contact with air was 26:1 at the time of the reaction. The damp box was sealed and allowed to stand at 25° C. for 60 minutes.

(1-3) Washing

The PET film was removed from a sample extracted from the damp box and then washed with pure water. The thus obtained samples are referred to as chip X1.

(1-4) 5-aminovaleric acid reaction

5-aminovaleric acid reaction was initiated within 1 hour after completion of the activation reaction. First, the chip X1 was dried well using an air gun. The chip X1 was set in a damp box, and then 200 μL of 1 M 5-aminovaleric acid solution was casted. A PET film having a thickness of 175 μm which was cut to have a size of 120 mm×8.5 mm was placed on the solution, so as to cover the surface while spreading the solution. The surface area ratio of the portion of the solution that was not in contact with air to the portion that was in contact with air was 24:1 at the time of the reaction. The damp box was sealed and allowed to stand at 25° C. for 90 minutes.

(1-5) Washing

The PET film was removed from a sample extracted from the damp box, and the sample was then washed with pure water. The thus obtained sample is referred to as chip X2.

(2) Pattern Formation for No-Ligand-Bound Portion

A pattern for a portion to which no ligand was able to bind was formed on the surface of the chip X2 by the following method. Specifically, at a specific position of the chip X2, PEG5000 (α-amino-ω-methoxy-polyethylene glycol) was covalently bound to the COOH group of 5-aminovaleric acid. PEG5000 is methoxy-terminated, so that it is unable to form covalent bonding with a ligand. Such portion was measured as a reference portion at the time of measuring the binding of analytes.

(2-1) Preparation of Reaction Solutions

20% PEG5000 solution: 4.5 g of PEG5000 was dissolved in 18.5 mL of extra pure water and 4 mL of 1 N NaOH.

0.2 M NHS solution: 2.32 g of NHS was dissolved in extra pure water to a volume of 100 mL.

0.4 M EDC solution: 7.7 g of EDC was dissolved in extra pure water to a volume of 100 mL.

(2-2) Patterning Reaction

The chip X2 was dried well using an air gun and then fixed on the seating of a dispenser (produced by Musashi Engineering Co., Ltd.). Next, a mixed solution (1 mL of 20% PEG5000 solution, 1 mL of 0.2 M NHS solution, and 2 mL of 0.4 M EDC solution) was poured into a syringe. 15 μL of the above mixed solution was spotted onto 6 different positions on the chip X2 at 18 mm intervals. The diameter of each droplet was approximately 4 mm. The chip X2 subjected to spotting was set in a damp box, the damp box was sealed, and then the damp box was allowed to stand at 25° C. for 60 minutes.

(2-3) Washing

Sample extracted from the damp box washed with 1 N citric acid aqueous solution and then further washed with pure water. The thus obtained sample is referred to as chip X3.

(3) PEG Linker Binding

A PEG linker (α-amino-ω-carboxyl-polyethylene glycol) was covalently bound to the COOH group of 5-aminovaleric acid existing on the surface of the chip X3 by the following method. The PEG linker does not bind to patterned surfaces.

(3-1) Preparation of Activation Solutions and Peg Linker Solution

0.1 M Sulfo-NHS solution: 2.04 g of NHS was dissolved in extra pure water to a volume of 100 mL.

0.4 M EDC solution: 7.7 g of EDC was dissolved in extra pure water to a volume of 100 mL.

10% PEG linker solution: 10 g of a PEG linker was dissolved in 80 mL of extra pure water and then the resultant was adjusted to pH8.5 using 1 N NaOH. Furthermore, extra pure water was added to the resultant to a volume of 100 mL.

(3-2) Activation

The chip X3 was dried well using an air gun. The chip X3 was then set in a damp box. 200 μL of a mixed solution (100 μL of 0.1 M Sulfo-NHS solution and 100 μL of 0.4 M EDC solution) was casted. A PET film having a thickness of 175 μm which was cut to have a size of 120 mm×8.5 mm was placed on the solution, so as to cover the surface while spreading the solution. The surface area ratio of the portion of the solution that was not in contact with air to the portion that was in contact with air was 26:1 at the time of the reaction. The damp box was sealed and allowed to stand at 25° C. for 60 minutes.

(3-3) Washing

The PET film was removed from a sample extracted from the damp box, and the sample was then washed with pure water. The thus obtained sample is referred to as chips X4.

(3-4) PEG Linker Reaction

A PEG linker reaction was initiated within 1 hour after completion of the activation reaction. First, the chip X4 was dried well using an air gun and then fixed on the seating of a dispenser (produced by Musashi Engineering Co., Ltd.). Next, 5 mL of 10% PEG linker solution was poured into a syringe. 8 μL of 10% PEG linker solution was spotted onto 6 different positions on the chip X4 at 18 mm intervals. A PET film having a thickness of 175 μm which was cut to have a size of 120 mm×8.5 mm was placed on the solution, so as to cover the surface while spreading the solution. The surface area ratio of the portion of the solution that was not in contact with air to the portion that was in contact with air was 90:1 at the time of the reaction. The sample was set in a damp box. The damp box was sealed and allowed to stand at 25° C. for 16 hours.

(3-5) Washing

The sample was extracted from the damp box and then washed with pure water. The thus obtained sample is referred to as chips X5.

(3-6) Storage

The chip X5 was dried well using an air gun and then stored.

### Test Example 3

Ligand protein was immobilized on 48 samples each of the above-treated chips XC and XF by the following method, and then variations in the amounts of the immobilized protein were evaluated. The amounts of the immobilized protein were measured using an SPR apparatus as shown in FIG. 3. A flow channel made of Tafthren (part 41 in FIG. 1) was used for measurement.

(1) Preparation of ligand solution: 0.5 mg of trypsin was dissolved in 1 ml of an acetate buffer (pH 5.5).

(2) Activation of sensor chip surface: NHS (0.1 M)/EDC (0.4 M)=1/1, 25° C., 30 minutes

(3) Immobilization of ligand: the above ligand solution, 25° C., 30 minutes

(4) Blocking: 1 M ethanol amine solution (pH 8.5), 25° C., 30 minutes

Variations in the amount of the immobilized protein are represented by CV values, and Table 2 shows the results. A CV value of 10% or more means insufficiency for practical use. It was shown that the chip of the present invention exhibits extremely good reproducibility of the amount of the immobilized protein.

TABLE 2

| | CV value of the amount of | |
|---|---|---|
| | immobilized protein | Remarks |
| Chip XC | 5.2 | Invention |
| Chip XF | 15.3 | Comparative |

### Example Y1

The sensor chip of the present invention was produced by the following method.

(1) Deposition of Gold Film onto Plastic Prism

A gold thin film was deposited on the upper surface of a plastic prism (FIG. 1) obtained by injection molding of ZEONEX (produced by ZEON CORPORATION) by the following method.

(1-1) Deposition of Gold Film

A prism was installed in a substrate holder of a sputtering apparatus (ULVAC SH-550), the inside of which was caused to become a vacuum (base pressure 1×10⁻³ Pa or less), and then Ar gas was introduced therein (1 Pa). DC power (4 kW) was applied to an 8-inch Au target for 1.5 seconds while keeping the substrate holder in a stationary state, thereby forming an Au thin film of 50 nm. An effective film thickness formation rate was approximately 33 nm/second. The thus obtained sample is referred to as chip YA.

(2) Polymer Coating

Polymer thin film was deposited on the gold thin films of chip YA by the following method.

(2-1) Preparation of Polymer Solution A

1.5 g of polymer (F-1) was dissolved in 100 mL of anhydrous MiBK (methyl isobutyl ketone), and then the resultant was filtered using a microfilter with a pore size of 0.45 μm. The moisture content of anhydrous MiBK was 20 ppm. 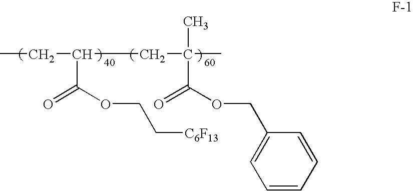
(2-2) Spin Coating

The chip YA was set in a spin coater (SC-408S sample seal-type spin coater, produced by a private limited company, Oshigane). The chip XA was fixed at a position 135 mm away from the center of the spin coater as shown in FIG. 2. 200 μL of a polymer solution A was casted so as to cover the entire gold film surface of the chip YA. Next, a wind-shielding cover was set so as to completely cover the chip YA, followed by spinning at 200 rpm for 60 seconds. After rotation was stopped, the chip YA was allowed to stand for 5 minutes.

(2-3) Vacuum Drying

The chip YA spin-coated with the polymer was vacuum-dried for 16 hours. The thus obtained samples are referred to as chip YB.

(3) Hydrolysis of Polymer Surface

The polymer thin film surface of the chip YB was hydrolyzed and then COOH groups were generated on the upper most surface by the following method.

(3-1) Hydrolysis

The chip YB was immersed in 1 N NaOH solution, followed by 16 hours of storage in a thermostatic bath at 60° C.

(3-2) Washing

After being extracted from the thermostatic bath at 60° C., the chip YB was naturally cooled for 15 minutes and then washed with extra pure water. The thus obtained samples are referred to as chips YC.

### Example Y2

A sensor chip was produced in a manner similar to that in Example Y1 except for performing Example Y (1-1), “Deposition of gold film” by the following method. The thus obtained sample is referred to as chip YD.

A prism was installed in a substrate holder of a sputtering apparatus (ULVAC SH-550), the inside of which was caused to become a vacuum (base pressure 1×10⁻³ Pa or less), and then Ar gas was introduced therein (1 Pa). DC power (4 kW) was applied to an 8-inch Au target for approximately 16 seconds while rotating (40 rpm) the substrate holder, thereby forming an Au thin film of approximately 50 nm. A metal film is deposited only when the substrate passes over the Au target. An effective film thickness formation rate was 33 nm/second.

### Example Y3

A sensor chip was produced in a manner similar to that in Example Y2 except for changing DC power to be applied to the Au target and adjusting application time so as to result in a film thickness of 50 nm. The thus obtained samples are referred to as chips YE and YF. Application times are shown in Table 3. The film thickness formation rates of chips YE and YF were 25 nm/second and 10 nm/second, respectively.

### Comparative Example Y1

A sensor chip was produced in a manner similar to that in Example Y2 except for changing DC power to be applied to an Au target and adjusting application time so as to result in a film thickness of 50 nm. The thus obtained samples are referred to as chips YG, YH, and YI. Application times are shown in Table 3. The film thickness formation rates of chips YG, YH, and YI were 5 nm/second, 2.5 nm/second, and 1.3 nm/second, respectively.

### Test Example Y1

### Determination of the Number of Separation

The number of separations was determined using chips YC to YF (of the present invention) and chips YG to YI (of comparative examples). Determination was performed by measuring the number of separations (cylindrically generated) in each of 5 visual fields using a ×20 light microscope and by obtaining average numbers thereof. Table 3 shows the results. In the case of the chips of the present invention, almost no separation took place. If the average number of separations is 5 or less, such chips are in an acceptable range in terms of marketability.

TABLE 3

| | | | Film | | |
|---|---|---|---|---|---|
| | | | deposition | | |
| | | | rate on | Average | |
| | Substrate | Application | substrate | number of | |
| | condition | time (sec) | (nm/sec) | separation | Remarks |
| Chip | Static | 1.5 | 33 | 0 | Invention |
| YC | | | | | |
| Chip | Rotating | 16 | 33 | 0 | Invention |
| YD | | | | | |
| Chip | Rotating | 21 | 25 | 0 | Invention |
| YE | | | | | |
| Chip | Rotating | 53 | 10 | 2 | Invention |
| YF | | | | | |
| Chip | Rotating | 106 | 5 | 38 | Comparative |
| YG | | | | | |
| Chip | Rotating | 212 | 2.5 | 53 | Comparative |
| YH | | | | | |
| Chip | Rotating | 424 | 1.3 | 60 | Comparative |
| YI | | | | | |

### Test Example Y2

### Surface Treatment of Chips

The surfaces of chips YC to YI were treated by the same method as that of the Test Example X2.

### Test Example Y3

Ligand protein was immobilized on 48 samples each of the above-treated chips YC to YI by the following method, and then variations in the amounts of the immobilized protein were evaluated. The amounts of the immobilized protein were measured using an SPR apparatus as shown in FIG. 3. A flow channel made of Tafthren (part 41 in FIG. 1) was used for measurement.

(1) Preparation of ligand solution: 0.5 mg of trypsin was dissolved in 1 ml of an acetate buffer (pH 5.5).

(2) Activation of sensor chip surface: NHS (0.1 M)/EDC (0.4 M)=1/1, 25° C., 30 minutes

(3) Immobilization of ligand: the above ligand solution, 25° C., 30 minutes

(4) Blocking: 1 M ethanol amine solution (pH 8.5), 25° C., 30 minutes

Variations in the amount of the immobilized protein are represented by CV values, and Table 4 shows the results. A CV value of 10% or more means insufficiency for practical use. It was shown that the chips of the present invention exhibit extremely good reproducibility of the amount of the immobilized protein.

TABLE 4

| | CV value of the amount of | |
|---|---|---|
| | immobilized protein | Remarks |
| Chip YC | 5.2 | Invention |
| Chip YD | 5.3 | Invention |
| Chip YE | 5.5 | Invention |
| Chip YF | 7.2 | Invention |
| Chip YG | 14.9 | Comparative |
| Chip YH | 16.3 | Comparative |
| Chip YI | 17.3 | Comparative |

### EFFECT OF THE INVENTION

The present invention enables the provision of a biosensor which exhibits good reproducibility of the amount of an immobilized physiologically active substance by suppressing plane deterioration (separation of a metal film from a resin) resulting from the formation of a thin metal film layer on a resin surface.

1. A biosensor which is produced by performing plasma irradiation on a light-reflecting surface of an optical block which is obtained by molding a resin and has at least 3 surfaces including a light incident surface, the light-reflecting surface and a light-emerging surface; forming a first metal layer; and then forming a second metal layer on the side of the first metal layer which is opposite to the light-reflecting surface.

2. The biosensor of claim 1 wherein the resin is a synthetic resin.

3. The biosensor of claim 1 wherein plasma irradiation is performed by using a plasma discharge gas that is selected from Ar, N₂, or O₂.

4. The biosensor of claim 1 wherein the light-reflecting surface of the optical block after plasma irradiation has surface roughness of Ra≦30 nm.

5. The biosensor of claim 1 wherein the first metal layer and the second metal layer are composed of different metals, respectively.

6. The biosensor of claim 1 wherein the metal layer is formed by a sputtering method.

7. The biosensor of claim 1 wherein the first metal layer and/or the second metal layer is/are formed with an adhesion rate of metal atoms to the light-reflecting surface that is 10 nm/second or greater.

8. The biosensor of claim 1 which is used for surface plasmon resonance analysis.

9. A biosensor which is produced by forming a metal layer on a light-reflecting surface of an optical block which is obtained by molding a resin and has at least 3 surfaces including a light incident surface, the light-reflecting surface and a light-emerging surface, with an adhesion rate of metal atoms to the reflecting surface that is 10 nm/second or greater.

10. The biosensor of claim 9 wherein the resin is a synthetic resin.

11. The biosensor of claim 9 wherein the metal layer is formed by a sputtering method.

12. The biosensor of claim 9 which is used for surface plasmon resonance analysis.

13. A method for producing the biosensor of claim 1, which comprises the steps of performing plasma irradiation on a light-reflecting surface of an optical block which is obtained by molding a resin and has at least 3 surfaces including a light incident surface, the light-reflecting surface and a light-emerging surface; forming a first metal layer on a plasma-irradiated light-irradiation surface; and then forming a second metal layer on the side of the first metal layer which is opposite to the light-reflecting surface.

14. A method for producing a biosensor of the present invention, which comprises the step of forming a metal layer on a light-reflecting surface of an optical block which is obtained by molding a resin and has at least 3 surfaces including a light incident surface, the light-reflecting surface and a light-emerging surface, with an adhesion rate of metal atoms to the reflecting surface that is 10 nm/second or greater.

15. The biosensor of claim 1, wherein a physiologically active substance is bound to the surface.

16. The biosensor of claim 9, wherein a physiologically active substance is bound to the surface.

17. A method for detecting or measuring a substance interacting with a physiologically active substance, which comprises a step of allowing a test substance to come into contact with the biosensor of claim 1 to the surface of which the physiologically active substance binds via a covalent bond.

18. The method of claim 17 wherein the substance interacting with the physiologically active substance is detected or measured by surface plasmon resonance analysis.

19. A method for detecting or measuring a substance interacting with a physiologically active substance, which comprises a step of allowing a test substance to come into contact with the biosensor of claim 9 to the surface of which the physiologically active substance binds via a covalent bond.

20. The method of claim 19 wherein the substance interacting with the physiologically active substance is detected or measured by surface plasmon resonance analysis.
